(19) United States
(12) Patent Application Publication
(10) Pub. No.: US 2023/0301700 A1
(43) Pub. Date: Sep. 28, 2023

(54) Treating Pulmonary Inflammatory Disease by Neural Ablation

(71) Applicant: Sorrento Therapeutics, Inc., San Diego, CA (US)

(72) Inventor: Alexis G. Nahama, San Diego, CA (US)

(73) Assignee: Sorrento Therapeutics, Inc., San Diego, CA (US)

(21) Appl. No.: 18/020,892

(22) PCT Filed: Aug. 10, 2021
(86) PCT No.: PCT/US2021/045367

§ 371 Date: Feb. 10, 2023

Related U.S. Application Data
(60) Provisional application No. 63/064,302, filed on Aug. 11, 2020.

(51) Int. Cl. A61B 18/06; A61B 18/14; A61K 41/00; A61K 31/05; A61K 31/045; A61K 31/047; A61K 33/14; A61K 31/055

(52) U.S. Cl. CPC A61B 18/06; A61B 18/14; A61K 41/0052; A61K 31/05; A61K 31/045; A61K 31/047; A61K 33/14; A61K 31/055; A61B 2018/0044

(57) ABSTRACT

There is disclosed a method for treating pulmonary inflammatory disease by neural ablation methods.

This application claims the benefit of priority of U.S. Provisional Patent Application No. 63/064,302, filed Aug. 11, 2020, which application is hereby incorporated by reference in its entirety. Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure provides a rescue therapy to interrupt a neurogenic inflammatory process occurring in the lungs and/or methods for treating pulmonary inflammatory disease, including pulmonary inflammatory disease associated with COVID-19. The methods disclosed comprise ablation of nerve fibers, e.g., in the vagus nerve, the stellate ganglion, a dorsal horn of the spinal cord, or a thoracic dorsal root ganglion.

### BACKGROUND

Neural ablation is a procedure in which a portion of nerve tissue is damaged, destroyed or removed to interrupt normal signaling pathways. Traditionally, ablation processes have been used to treat pain or to control arrhythmias in patients with heart disease.

Neural ablation may be accomplished using chemicals such as neurolytic agents, that can be delivered, e.g., epidurally, peri-ganglionically via nerve block, intra-ganglionically, or by local infiltration. In some instances, the chemical treatment can be monitored or advanced using ultrasound imaging. In other instances, neural ablation may be accomplished by radiofrequency ablation (RFA) or pulsed RFA processes which cause neural damage using heat.

Coronaviruses are a group of viruses that causes diseases in birds, mammals and humans. The diseases include respiratory infections and enteric infections which can be mild or lethal. Coronaviruses are viruses in the subfamily Orthocoronavirinae, in the family Coronaviridae, in the order Nidovirales. The genus Coronavirus includes avian infectious bronchitis virus, bovine coronavirus, canine coronavirus, human coronavirus 299E, human coronavirus OC43, murine hepatitis virus, rat coronavirus, and porcine hemagglutinating encephalomyelitis virus. The genus Torovirus includes Berne virus and Breda virus. Coronaviruses are enveloped viruses having a positive-sense single-stranded RNA genome and a nucleocapsid of helical symmetry. The genomic size of coronaviruses ranges from approximately 26 to 32 kilobases, which is believed to be the largest for an RNA virus. It is interesting to note that the 2019-2020 China pneumonia outbreak in Wuhan was traced to a novel coronavirus, labeled 2019-nCoV by the World Health Organization (WHO), and also known as SARS-CoV-2, which causes Coronavirus disease 2019, or COVID-19.

Respiratory failure due to acute respiratory distress syndrome (ARDS) is one of the major causes of mortality (53%) associated with COVID-19 disease (Ruan et al. (2020) Intensive Care Med Mar 3:10-3). Around 10% of the patients require intensive care unit (ICU) care with ventilatory support and an ICU mortality rate of 79% has been reported (Huang et al. (2020) Lancet Vol. 394, Issue 10233, P497-506). ARDS was first described in 1967 (Ashbaugh et al. (1967) Lancet 2:319-323) and is characterized by diffuse pulmonary microvascular injury resulting in increased permeability and hypoxemia caused by intrapulmonary shunts. The first two stages of ARDS progression (i.e., 12-72 hours after onset) can represent a critical window for intervention as the syndrome can be reversed if the initiating factors and the inflammatory mediators can be controlled. An early diagnosis may also be facilitated if the initiating stimulus is known as in determination of sepsis, aspiration of gastric contents, multiple transfusions, severe fractures, burns, pancreatitis or severe trauma. Upon progression to a third stage of ARDS pulmonary hypertension increases, heart rate increases to compensate for hypoxemia and mechanical ventilation supportive therapy is generally required. Pathologically the cellular infiltrates are denser with continued neutrophil infiltration and increasing mononuclear, lymphocyte and fibroblast cell infiltrates.

The severity of the disease is higher in older patients with 80% death observed in those over 60-65 years of age (CDC COVID-19 Response Team (2020) MMWR Morb Mortal Wkly Rep 69:343-346), while younger infected seem to be less susceptible and exhibit medium-mild symptoms (Wu et al. (2020) JAMA Published online Feb. 24, 2020). Once the lower respiratory tract is affected, the respiratory distress progresses very quickly, with time to death reported as rapidly as 14 days from initial symptoms despite availability of ventilator palliative support. It has been proposed that the severity and mortality rates of the susceptible population infected by COVID-19 is related to a cytokine storm, in which an exaggerated production of pro-inflammatory substances are released into the pulmonary microenvironment over a short period of time (Mehta et al. (2020) Lancet Vol. 395, Issue 10229, P1033-1034).

Novel life-saving strategies are desperately needed to mitigate the high mortality that is associated with the late stage viral infection with acute respiratory distress.

### SUMMARY

The present disclosure provides a method for treating pulmonary inflammatory diseases, including viral, bacterial or chemical insult to the lungs triggering an initial inflammatory process that is exacerbated by the immune system stimulated by the neural pathways, comprising neural ablation, such as of the stellate ganglion, vagal nerve, dorsal horn of the spinal cord, or thoracic dorsal root ganglion.

Embodiment 1 is a method for treating pulmonary inflammatory disease and/or interrupting a neurogenic inflammatory process occurring in a lung in a subject, comprising ablating a vagal nerve, stellate ganglion, dorsal horn of the spinal cord, or thoracic dorsal root ganglion of the subject by chemical ablation or radiofrequency ablation.

Embodiment 2 is the method of embodiment 1, wherein the vagal nerve is ablated.

Embodiment 3 is the method of embodiment 1, wherein the stellate ganglion is ablated.

Embodiment 4 is the method of embodiment 1, wherein the thoracic dorsal root ganglion is ablated.

Embodiment 5 is the method of embodiment 1, wherein a dorsal horn of the spinal cord is ablated.

Embodiment 6 is the method of any one of embodiments 1 to 5, wherein the ablating is by radiofrequency ablation.

Embodiment 7 is the method of embodiment 6, wherein the radiofrequency ablation comprises application of alternating current with a frequency of about 350-500 kHz.

Embodiment 8 is the method of any one of embodiments 1 to 5, wherein the ablating is by chemical ablation.

Embodiment 9 is the method of embodiment 8, wherein the chemical ablation comprises administering to the subject an effective amount of a neurolytic agent epidurally, peri-ganglionically, intra-ganglionically or by local infiltration.

Embodiment 10 is the method of embodiment 9, wherein the neurolytic agent comprises phenol, chlorocresol, ethanol, or glycerol.

Embodiment 11 is the method of embodiment 9, wherein the neurolytic agent comprises hypertonic saline.

Embodiment 12 is the method of embodiment 9, wherein the neurolytic agent comprises a neurotoxin.

Embodiment 13 is the method of any one of embodiments 1 to 12, wherein the subject is an adult human.

Embodiment 14 is the method of any one of embodiments 1 to 13, wherein the method comprises epidural administration.

Embodiment 15 is the method of any one of embodiments 1 to 13, wherein the method comprises a peri-ganglionic nerve block.

Embodiment 16 is the method of any one of embodiments 1 to 13, wherein the method comprises intra-ganglionic administration.

Embodiment 17 is the method of any one of embodiments 1 to 13, wherein the method comprises local infiltration.

Embodiment 18 is the method of any one of embodiments 8 to 17, wherein the neurolytic agent is administered in a pharmaceutical formulation comprising the neurolytic agent and a pharmaceutically acceptable carrier.

Embodiment 19 is the method of any one of embodiments 1 to 18, wherein the pulmonary inflammatory disease comprises acute respiratory distress syndrome (ARDS), chronic obstructive pulmonary disease (COPD), pulmonary arterial hypertension (PAH), chronic inflammatory lung disease, pulmonary fibrosis, pulmonary vasculitis, pulmonary sarcoidosis, inflammation and/or infection associated with lung transplantation, acute or lung rejection and/or dysfunction, bronchitis, sinusitis, asthma, cystic fibrosis, bacterial infection, fungal infection, parasite infection, viral infection, bronchiolitis obliterans syndrome (BOS), primary ciliary dyskinesia (PCD), alveolar proteinosis, idiopathic pulmonary fibrosis (IPF), eosinophilic pneumonia, eosinophilic bronchitis, inflammation and/or infection associated with mechanical ventilation, ventilator-associated pneumonia, asbestos-related airway disorder or disease, dust-related airway disorder or disease, silicosis, or radiation or chemical agent-related airway disease or disorder, or any combination thereof.

Embodiment 20 is the method of any one of embodiments 1 to 19, wherein the pulmonary inflammatory disease comprises acute respiratory distress syndrome (ARDS).

Embodiment 21 is the method of any one of embodiments 1 to 19, wherein the pulmonary inflammatory disease comprises chronic obstructive pulmonary disease (COPD).

Embodiment 22 is the method of any one of embodiments 1 to 21, wherein the pulmonary inflammatory disease comprises pulmonary arterial hypertension (PAH).

Embodiment 23 is the method of any one of embodiments 1 to 22, wherein the pulmonary inflammatory disease comprises inflammation and/or infection associated with mechanical ventilation and/or ventilator-associated pneumonia.

Embodiment 24 is the method of any one of embodiments 1 to 23, wherein the pulmonary inflammatory disease is associated with viral pneumonia, influenza, or a coronavirus infection.

Embodiment 25 is the method of any one of embodiments 1 to 24, wherein the pulmonary inflammatory disease is associated with COVID-19.

Embodiment 26 is the method of any one of embodiments 1 to 25, further comprising ablating afferent nerves in the thoracic dorsal root ganglion.

Embodiment 27 is the method of embodiment 26, wherein ablating afferent nerves in the thoracic dorsal root ganglion supports palliative ventilation therapy.

Embodiment 28 is the method of any one of embodiments 1 to 27, wherein chemical ablation or radiofrequency ablation is administered once in a single dose.

Embodiment 29 is the method of any one of embodiments 1 to 27, wherein chemical ablation or radiofrequency ablation is administered periodically.

Embodiment 30 is the method of any one of embodiments 1 to 29, wherein the subject has pulmonary inflammatory disease.

Embodiment 31 is the method of any one of embodiments 1 to 30, wherein the subject has a neurogenic inflammatory process occurring in a lung.

Embodiment 32 is a neurolytic agent or radiofrequency source for use in the method of any one of embodiments 1 to 31.

Embodiment 33 is a use of a neurolytic agent or radiofrequency source for the manufacture of a medicament for use in the method of any one of embodiments 1 to 31.

### DETAILED DESCRIPTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like. It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In some embodiments, “about” encompasses variation within 10%, 5%, 2%, 1%, or 0.5% of a stated value.

Numeric ranges are inclusive of the numbers defining the range. Measured and measurable values are understood to be approximate, taking into account significant digits and the error associated with the measurement. Also, all ranges are to be interpreted as encompassing the endpoints in the absence of express exclusions such as “not including the endpoints”; thus, for example, “ranging from 1 to 10” includes the values 1 and 10 and all integer and (where appropriate) non-integer values greater than 1 and less than 10.

The use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings. Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

As used herein, “ablation” or “neural ablation” refers to the removal, destruction, or inactivation of a part of a biological tissue (e.g., vagal nerve or stellate ganglion), and may be carried out by chemicals (chemical ablation or chemoablation), or electricity (Radiofrequency ablation or fulguration). To be clear, ablation of, e.g., a vagal nerve or stellate ganglion does not refer to the complete destruction thereof.

As used herein, “chemical ablation” refers to the injection of a chemical or chemical mixture at or near a nerve ending to cause neurolysis.

As used herein, “ultrasound-guided sclerotherapy” refers to chemical ablation performed with guidance from observation via ultrasound imaging. The procedure allows for precise and minimally invasive treatments.

As used herein, “cytokine storm” or hypercytokinemia, refers to the severe immune reaction in which the body releases cytokines into the blood too quickly. A cytokine storm can occur as a result of an infection (e.g., a coronavirus infection). Signs and symptoms may include high fever, inflammation (redness and swelling), and severe fatigue and nausea. A cytokine storm may be severe or life threatening and lead to multiple organ failure. Cytokine storms have been associated with the Sars-CoV-2 virus and symptoms associated Covid-19.

As used herein, “pulmonary inflammatory disease” is used collectively to refer to those acute and chronic pathological conditions associated with inflammatory processes. Non-limiting examples of pulmonary inflammatory disease includes acute respiratory distress syndrome (ARDS), pneumonia, pneumonitis, bronchitis, lung infections, atelactasis, conditions associated with inflammatory lung injuries such as chemotherapeutic (e.g., bleomycin) induced lung injury, pancreatitis induced lung injury, hyperoxia induced lung injury, amiodarone induced pneumonitis, radiation pneumonitis, chlorine gas or smoke inhalation injuries, bronchiolitis obliterans/obstructive pneumonia (BOOP), viral and mycoplasmal pneumonias (e.g., Legionella and CMV lung), pneumoconioses, pulmonary vasculitis, pulmonary sarcoidosis, airways bacterial infection, airways fungal infection, airways parasite infection, airways viral infection, mechanical ventilation-associated inflammation and/or infection, ventilator-associated pneumonias. Non-limiting examples of chronic pathological conditions of the lung include chronic obstructive pulmonary disease (COPD), pulmonary arterial hypertension (PAH), cystic fibrosis, silicosis, asbestosis, asthma, atherosclerosis, chronic bronchitis, chronic inflammation due to chronic bacterial or viral infections, coronary artery disease, idiopathic pulmonary fibrosis (IPF), familial pulmonary fibrosis (FPF), desquamative interstitial pneumonitis (DIP), hypersensitivity pneumonitis, interstitial pneumonitis, collagen vascular disease, sarcoidosis, coal worker's pneumoconiosis, bronchopulmonary dysplasia, inflammatory pseudotumor. As used herein, “neurogenic inflammatory process” refers to a process by which central stimulation of sensory nerves elicits antidromic impulses causing vasodilatation, plasma extravasation, and other inflammatory changes in peripheral tissue. The neurogenic inflammation is initiated by activation of peripheral nervous system c-fiber neurons rather than by immunological events. The neuronal activity leads to neuropeptide release and inflammation at sites different from the original stimulus.

As used herein, “epidural administration” refers to delivery of a drug or pharmaceutical formulation into the epidural space (also known as “extradural space” or “peridural space”) which is the outermost part of the spinal canal. It is the space within the canal (formed by the surrounding vertebrae) lying outside the dura mater (which encloses the arachnoid mater, subarachnoid space, the cerebrospinal fluid, and the spinal cord). For example, epidural delivery may include delivery to the epidural space without direct injection into nerves or may include epidural delivery into nerve tissue.

As used herein, a “nerve block” refers to an administration of an agent (e.g., a medication or a neurolytic agent), around a specific nerve or a bundle of nerves, such that the agent prevents transmission of impulses through the nerves.

As used herein, “neurolysis” refers to the application of physical or chemical agents to a nerve in order to cause a degeneration of targeted nerve fibers. When the nerve fibers degenerate, it causes an interruption in the transmission of nerve signals.

As used herein, “neurolytic agent” refers to a chemical agent such as alcohol, phenol, glycerol, an ammonium salt such as ammonium chloride, an aminoglycoside such as streptomycin or gentamicin, chlorocresol, hypertonic saline, a hypotonic solution, or a neurotoxin, that can be used to ablate nerve fibers.

As used herein, “peri-ganglionic administration” refers to delivery of a drug or pharmaceutical formulation into the vicinity of a ganglion.

As used herein, “administration by local infiltration” refers to delivery of a drug or pharmaceutical formulation by injection so as to affect nervous tissue in a limited area.

As used herein, “radiofrequency ablation” (RFA), also called fulguration, refers to an ablation process using the heat generated from medium frequency alternating current (e.g., in the range of 350-500 kHz). Radio frequency current does not directly stimulate nerves.

As used herein “stellate ganglion” refers to the collection of nerves (sympathetic) found at the level of the sixth and seventh cervical vertebrae (the last vertebra of the neck). The nerves are located in front of the vertebrae. These nerves are part of the sympathetic nervous system and supply the face and arm but are not involved with feeling or movement.

As used herein “vagus nerve” or “vagal nerve” refers to the X cranial nerve or 10th cranial nerve, the longest and most complex of the cranial nerves, running from the brain through the face and thorax to the abdomen. It is a mixed nerve that contains parasympathetic fibers. The vagus has cardiac, esophageal, and pulmonary branches.

As used herein, “dorsal horn of the spinal cord” refers to the grey matter section of the spinal cord that receives several types of sensory information from the body including light touch, proprioception, and vibration. This information is sent from receptors of the skin, bones, and joints through sensory neurons whose cell bodies lie in the dorsal root ganglion.

As used herein, “thoracic dorsal root ganglion” refers to a cluster of neurons (a ganglion) in a dorsal root of a spinal nerve located in the thoracic region of the spine. The dorsal root is the afferent sensory root and carries sensory information from the skin, muscles, and visceral organs to the brain. The root terminates in dorsal root ganglion, which is composed of the cell bodies of the corresponding neurons.

“Intra-ganglionic administration” means administration to a ganglion. Intra-ganglionic administration can be achieved by direct injection into the ganglion and also includes selective nerve root injections, in which the compound passes up the connective tissue sleeve around the nerve and enters the ganglion from the nerve root just outside the vertebral column.

The terms “effective amount”, “therapeutically effective amount” or “effective dose” or related terms may be used interchangeably and refer to an amount of the therapeutic agent that when administered to a subject, is sufficient to affect a measurable improvement or prevention of a disease or disorder associated with coronavirus infection. For example, administering an effective dose sufficient to inhibit the proliferation and/or replication of the coronavirus, and/or the development of the viral infection within the subject. Therapeutically effective amounts of the therapeutic agents provided herein, when used alone or in combination with an antiviral agent, will vary depending upon the relative activity of the therapeutic agent, and depending upon the subject and disease condition being treated, the weight and age and sex of the subject, the severity of the disease condition in the subject, the manner of administration and the like, which can readily be determined by one of ordinary skill in the art. In one embodiment, a therapeutically effective amount will depend on certain aspects of the subject to be treated and the disorder to be treated and may be ascertained by one skilled in the art using known techniques. In addition, as is known in the art, adjustments for age as well as the body weight, general health, sex, diet, time of administration, drug interaction, and the severity of the disease may be necessary.

The terms “subject” and “patient” as used herein refer to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of a therapeutic agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, by local infiltration, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treating” is to be understood broadly and encompasses any beneficial effect, including, e.g., delaying, slowing, or arresting the worsening of symptoms associated with pulmonary inflammatory disease or remedying such symptoms, at least in part. Treating also encompasses bringing about any form of improved patient function, as discussed in detail below. In some embodiments, treatment also means prolonging survival as compared to expected survival if not receiving treatment. Those in need of treatment include those who already have the disease or disorder, as well as those who tend to have the disease or disorder or who should prevent the disease or disorder.

A “pharmaceutically acceptable vehicle” for therapeutic purposes is a physical embodiment that can be administered to a subject. Pharmaceutically acceptable vehicles include pills, capsules, caplets, tablets, oral fluids, injection fluids, sprays, aerosols, troches, dietary supplements, creams, lotions, oils, solutions, pastes, powders, steam, or it may be a liquid, but is not limited to these. An example of a pharmaceutically acceptable vehicle is a buffered isotonic solution such as phosphate buffered saline (PBS).

### Overview

Some viral, bacterial or chemical insults to the lungs have been observed to cause an initial inflammatory process which may lead to a cytokine storm. For example, the clinical signs of COVID-19 are consistent with those observed in viral pneumonia, both of which can progress to onset of ARDS. These pulmonary changes are likely responsible for both systemic and localized immune response leading to a hyperinflammatory state. The mortality rate in patients is suspected to be related to virally driven cytokine storm similar to that seen in SARS-CoV-2 infections.

The cytokine storm is a result of a severe immune reaction, for example in the lungs, as measured by high levels of inflammatory markers (c-reactive protein, serum ferritin) and cytokine levels (IL-6, IL-2, IL-7, IL-10, GSCF, IP10, MCP1, MIP1A, and TNFα) in the plasma. ICU patients have higher plasma levels of IL-2, IL-7, IL-10, GSCF, IP10, MCP1, MIP1A, and TNFα as compared to non-ICU patients, indicating that the presence of high circulating cytokine levels is associated with the severity of the disease. It is therefore necessary to interfere with the inflammatory cascade at a higher level (i.e., eliminating the pro-inflammatory efferent pathway) to appropriately control the multimodal aspect of this inflammatory process.

The morbidity, severity of the disease, and underlying physiological events linked to mortality can be explained by the involvement of the TRPV1 expressing neuronal system (afferent/efferent neurons). TRPV1, the transient receptor potential cation channel subfamily V member 1 (also known as Vanilloid receptor-1 (VR1)) is a multimeric cation channel prominently expressed in nociceptive primary afferent neurons (Caterina et al. (1997) Nature 389:816-824; Tominaga et al. (1998) Neuron 21:531-543).

Sensory neurons innervating the heart and lung enter the central nervous system by one of two routes; through the vagus nerve into the brain stem (medulla) with cell bodies residing in the nodose ganglia and directly into the spinal cord where cell bodies reside in the Dorsal Root Ganglia (DRG). Afferents are composed of elements that respond to a variety of sensory modalities including, but not limited to, mechanical deformation, heat, cold, pH, and inflammatory mediators. The reflex effects following stimulation of these afferents depends on the type of stimulus and the neural pathway involved. Activation of vagal afferent pathways tends to be sympatho-inhibitory and anti-inflammatory, while activation of spinal afferents tends to be sympatho-excitatory and pro-inflammatory.

Both vagal and spinal afferent fibers are composed of A-fiber (high conduction velocity) and C-fiber (low conduction velocity) axons. These fibers and their sensory endings express a variety of membrane receptors that mediate ion channel function including traditional Na, K and Ca channels (both voltage gated and ligand gated). Non-specific cation channels that are highly permeable to calcium are expressed. These include at least 30 members of the Transient Receptor Potential family including Transient Receptor Potential A (TRPA) and Transient Receptor Vanilloid (TRPV) receptors. TRPV1 receptors transduce sensations of heat and neuropathic pain in the periphery. It has been widely reported that activation of TRPV1-expressing afferents causes secretion of neuropeptides such as substance P (SP) and calcitonin gene-related peptide (CGRP) (See Nicoletti et al. (2012) Int J Immunopathol Pharmacol 25(4):849-57; Bhatia (2010) Antioxid Redox Signal 12(10):1191-202; Fernandes et al. (2009) Handb Exp Pharmacol 194:393-416; Scardina et al. (2004) Minerva Stomatol 53 (1-2):21-32; Harrison et al.(2001) Int J Biochem Cell Biol 33(6):555-76). Released SP, but not CGRP, in sensory endings binds neurokinin (NK) 1 receptors on blood vessels causes vasodilation and increased vascular permeability that allows loss of proteins and fluid (plasma extravasation) thus promoting the regional accumulation of monocytes and leukocytes contributing to inflammation (See Roberts et al. (2004) Brain Res 995(2):176-83; Andrews et al. (1989) Br J Pharmacol 97(4):1232-8; and McConalogue et al. (1998) Mol Biol Cell 9(8):2305-24). In the lung, this can cause pulmonary edema resulting in reduced oxygen diffusion.

It is recognized herein that ablation of pulmonary TRPV1-containing afferents can provide a therapeutic strategy for treating pulmonary inflammatory disease, e.g., respiratory distress syndrome (ARDS).

Various neurolytic agents or the technique of radiofrequency ablation can be used to ablate TRPV1-expressing neurons in dorsal root ganglia (DRG), dorsal horns (DH) of the spinal cord, or peripheral nerve endings. Disclosed herein is the use of ablating agents against TRPV1 positive pulmonary pathways in patients with acute pulmonary inflammatory disease. Such a therapeutic approach targeting TRPV1 expressing neurons in the lungs may modulate the inflammatory and immune signal activity, leading to reduced mortality and better overall outcomes.

### Exemplary Methods and Compositions for Use

Provided herein are compositions and methods and procedures for interrupting a neurogenic inflammatory process occurring in a lung and/or treating pulmonary inflammatory disease using neural ablation procedures. In some embodiments the neural ablation targets the stellate ganglion, the vagal nerve, the dorsal horn of the spinal cord, or the thoracic dorsal root ganglion. In some embodiments the neural ablation targets the stellate ganglion. In some embodiments the neural ablation targets the vagal nerve. In some embodiments the neural ablation targets the dorsal horn of the spinal cord. In some embodiments the neural ablation targets the thoracic dorsal root ganglion. In some embodiments, the neural ablation blocks progression of a cytokine storm, e.g., thus interrupting or calming the immune system's overreaction.

Provided herein are neural ablation methods for treating pulmonary inflammatory disease wherein the neural ablation method is chosen from radiofrequency ablation and chemical ablation. Provided herein are neural ablation methods for interrupting a neurogenic inflammatory process occurring in a lung wherein the neural ablation method is chosen from radiofrequency ablation and chemical ablation. In some embodiments the ablation process is radiofrequency ablation of nerve fibers. In some embodiments the ablation process is a chemical ablation procedure. In some embodiments, the ablation process is a chemical neurolysis which may cause deconstructive fibrosis which then disrupts the sympathetic ganglia, an effect that may last for three to six months. In some embodiments the nerve fibers are located in the vagal nerve, the stellate ganglion, the dorsal horn of the spinal cord, or the thoracic dorsal root ganglion. In some embodiments the nerve fibers are located in the vagal nerve. In some embodiments the nerve fibers are located in the stellate ganglion. In some embodiments the nerve fibers are located in the dorsal horn of the spinal cord. In some embodiments the nerve fibers are located in the thoracic dorsal root ganglion.

Provided herein are methods of treating and compositions for use in interrupting a neurogenic inflammatory process occurring in a lung and/or treating pulmonary inflammatory disease using chemical ablation procedures in which a neurolytic agent is delivered epidurally, peri-ganglionically via nerve block, intra-ganglionically or by local infiltration. In some embodiments, the neurolytic agent is delivered to the nerve fibers in the vagal nerve, the thoracic dorsal root ganglion, the dorsal horn of the spinal cord, or the stellate ganglion. In some embodiments, the neurolytic agent is delivered to the nerve fibers in the vagal nerve. In some embodiments, the neurolytic agent is delivered to the nerve fibers in the stellate ganglion. In some embodiments, the neurolytic agent is delivered to the nerve fibers in the dorsal root ganglion. In some embodiments, the neurolytic agent is delivered to the nerve fibers in the dorsal horn of the spinal cord. In various embodiments, the route of administration for a neurolytic agent includes administration by local infiltration, thoracic epidural injections, peri-ganglionic nerve block or intra-ganglionic injections for “chemical” targeted lung denervation. In one embodiment, a neurolytic agent is administered by accessing the vagal nerve with a local ablative agent through the neck, going low and away from the carotid bulb. The nerve location could then be confirmed using ultrasound guidance. In one embodiment, a neurolytic agent is administered by accessing the stellate ganglion.

In some embodiments the neurolytic agent is chosen from glycerol, phenol, ethanol or a neurotoxin. In some embodiments the neurolytic agent is glycerol. In some embodiments the neurolytic agent is phenol. In some embodiments the neurolytic agent is ethanol. In some embodiments the neurolytic agent is a neurotoxin.

In some embodiments, an epidural, intraganglionic, or peri-ganglionic injection of neurolytic agent in subjects with advanced COVID-19 disease supports palliative ventilation therapy by ablating afferent nerves at the thoracic dorsal root ganglion (DRG) level to increase survival.

The methods described herein are for use with any subject in whom the neurolytic agent is effective, e.g., able to ablate the vagal nerve, stellate ganglion, the dorsal horn of the spinal cord, or the thoracic dorsal root ganglion, and who is in need of treatment for PD. In some embodiments, the neurolytic agent is administered at doses typical for ablation processes and that are neurotoxic. In some embodiments, a 2-, 3-, or 4-point peri-ganglionic nerve block technique is used. In some embodiments, a 2-point peri-ganglionic nerve block technique is used. In some embodiments, a 3-point peri-ganglionic nerve block technique is used. In some embodiments, a 4-point peri-ganglionic nerve block technique is used.

The dosage can be adjusted depending on the proximity of the site of administration to the nerve fiber. For example, where ultrasound or a nerve stimulator is used to ensure that the site of administration is very close to the nerve, a lower dose and/or volume can be used. Alternatively, a nerve block can be accomplished using a larger volume to ensure contact with the desired nerves. Notably, neurolytic agents specific for the TRPV1 receptor would not affect non-target nerves such as motor neurons that do not have enough TRPV1 receptors to be sensitive to the neurolytic agent.

In some embodiments, the neurolytic agent, at doses typical for ablation processes and that are neurotoxic, is administered with a pharmaceutically acceptable carrier. In some embodiments, the pharmaceutically acceptable carrier comprises water. In some embodiments, the pharmaceutically acceptable carrier comprises any one or more of polysorbate 80, polyethylene glycol, a sugar or sugar alcohol (e.g., mannitol or dextrose), a pharmaceutically acceptable buffer (e.g., phosphate buffer), and/or a pharmaceutically acceptable salt (e.g., NaCl). In some embodiments, the pharmaceutically acceptable carrier comprises an organic solvent such as ethanol or DMSO, e.g., as a minority or residual component used as an aid in dissolving neurolytic agent before dilution in a primarily aqueous composition.

The concentration of neurolytic agent in the formulation may be any suitable value for delivery of the intended dose. Appropriate concentrations of various neurolytic agents are known in the art. For example, an ammonium salt such as ammonium chloride may be delivered at a concentration of about 2% by weight. Ethanol may be delivered at about 45-100% or 45-95% by volume. Phenol may be delivered at about 5-15% by weight or about 5-7% by weight. Chlorocresol may be used at a concentration of about 2-2.5% by weight. Hypertonic saline may be used at a concentration of about 10% NaCl by weight. For exemplary discussions of neurolytic agent concentration and administration, see, e.g., Swerdlow, Anaesthesia 33:733-40 (1978); Manchikanti et al., Pain Physician 4:366-73 (2001).

In some embodiments, the neurolytic agent may be administered as a one-time single dose. In some embodiments, the neurolytic agent may be periodically administered. In some embodiments, the neurolytic agent may be periodically administered to a subject in need of treatment for pulmonary inflammatory disease as needed to reduce the severity of the disease.

Provided herein are composition and methods for interrupting a neurogenic inflammatory process occurring in a lung and/or treating pulmonary inflammatory disease, comprising administering the neurolytic agent to a subject via epidural, peri-ganglionic, intra-ganglionic injection or by local infiltration. One embodiment provides a method of treating a mammalian subject suffering from ARDS.

In some embodiments, radiofrequency ablation may be administered as a one-time single dose. In some embodiments, radiofrequency ablation may be periodically administered. In some embodiments, radiofrequency ablation may be periodically administered to a subject in need of treatment for pulmonary inflammatory disease as needed to reduce the severity of the disease. In some embodiments, radiofrequency ablation is periodically administered to a subject in need of interrupting a neurogenic inflammatory process occurring in a lung as needed. Any suitable radiofrequency source can be used to apply electricity to achieve ablation in the methods described herein.

Provided herein are methods for interrupting a neurogenic inflammatory process occurring in a lung and/or treating pulmonary inflammatory disease, comprising administering radiofrequency ablation to a subject. One embodiment provides a method of treating a mammalian subject suffering from ARDS.

In exemplary embodiments, the neural ablation methods disclosed herein may be administered to reduce the patient's symptoms or can be administered to counter the mechanism of the disease itself. It will be appreciated by those skilled in the art that these therapeutic objectives are often related and the treatment can be adjusted for individual patients based on various factors. These factors include the patient's age, gender, or health status, progression of pulmonary inflammatory disease, degree of dyspnea, amount of tissue damage to the patient's respiratory tract, patient smoking history, and various environmental factors (e.g., temperature, humidity and air pollution), which may contribute to the patient's condition. The patient's therapy can be adjusted depending on the dosage, timing, route of administration, and by administering other therapeutic agents simultaneously or sequentially.

The complete disclosures of all publications cited herein are incorporated herein by reference in their entireties as if each were individually set forth in full herein and incorporated.

Various modifications and alterations to the embodiments disclosed herein will become apparent to those skilled in the art without departing from the scope and spirit of this disclosure. Illustrative embodiments and examples are provided as examples only and are not intended to limit the scope of the present invention.

1. A method for treating pulmonary inflammatory disease and/or interrupting a neurogenic inflammatory process occurring in a lung in a subject, comprising ablating a vagal nerve, stellate ganglion, dorsal horn of the spinal cord, or thoracic dorsal root ganglion of the subject by chemical ablation or radiofrequency ablation.

2. The method of claim 1, wherein the vagal nerve is ablated.

3. The method of claim 1, wherein the stellate ganglion is ablated.

4. The method of claim 1, wherein the thoracic dorsal root ganglion is ablated.

5. The method of claim 1, wherein a dorsal horn of the spinal cord is ablated.

6. The method of any one of claims 1 to 5, wherein the ablating is by radiofrequency ablation.

7. The method of claim 6, wherein the radiofrequency ablation comprises application of alternating current with a frequency of about 350-500 kHz.

8. The method of any one of claims 1 to 5, wherein the ablating is by chemical ablation.

9. The method of claim 8, wherein the chemical ablation comprises administering to the subject an effective amount of a neurolytic agent epidurally, peri-ganglionically, intra-ganglionically or by local infiltration.

10. The method of claim 9, wherein the neurolytic agent comprises phenol, chlorocresol, ethanol, or glycerol.

11. The method of claim 9, wherein the neurolytic agent comprises hypertonic saline.

12. The method of claim 9, wherein the neurolytic agent comprises a neurotoxin.

13. The method of any one of claims 1 to 12, wherein the subject is an adult human.

14. The method of any one of claims 1 to 13, wherein the method comprises epidural administration.

15. The method of any one of claims 1 to 13, wherein the method comprises a peri-ganglionic nerve block.

16. The method of any one of claims 1 to 13, wherein the method comprises intra-ganglionic administration.

17. The method of any one of claims 1 to 13, wherein the method comprises local infiltration.

18. The method of any one of claims 8 to 17, wherein the neurolytic agent is administered in a pharmaceutical formulation comprising the neurolytic agent and a pharmaceutically acceptable carrier.

19. The method of any one of claims 1 to 18, wherein the pulmonary inflammatory disease comprises acute respiratory distress syndrome (ARDS), chronic obstructive pulmonary disease (COPD), pulmonary arterial hypertension (PAH), chronic inflammatory lung disease, pulmonary fibrosis, pulmonary vasculitis, pulmonary sarcoidosis, inflammation and/or infection associated with lung transplantation, acute or lung rejection and/or dysfunction, bronchitis, sinusitis, asthma, cystic fibrosis, bacterial infection, fungal infection, parasite infection, viral infection, bronchiolitis obliterans syndrome (BOS), primary ciliary dyskinesia (PCD), alveolar proteinosis, idiopathic pulmonary fibrosis (IPF), eosinophilic pneumonia, eosinophilic bronchitis, inflammation and/or infection associated with mechanical ventilation, ventilator-associated pneumonia, asbestos-related airway disorder or disease, dust-related airway disorder or disease, silicosis, or radiation or chemical agent-related airway disease or disorder, or any combination thereof.

20. The method of any one of claims 1 to 19, wherein the pulmonary inflammatory disease comprises acute respiratory distress syndrome (ARDS).

21. The method of any one of claims 1 to 19, wherein the pulmonary inflammatory disease comprises chronic obstructive pulmonary disease (COPD).

22. The method of any one of claims 1 to 21, wherein the pulmonary inflammatory disease comprises pulmonary arterial hypertension (PAH).

23. The method of any one of claims 1 to 22, wherein the pulmonary inflammatory disease comprises inflammation and/or infection associated with mechanical ventilation and/or ventilator-associated pneumonia.

24. The method of any one of claims 1 to 23, wherein the pulmonary inflammatory disease is associated with viral pneumonia, influenza, or a coronavirus infection.

25. The method of any one of claims 1 to 24, wherein the pulmonary inflammatory disease is associated with COVID-19.

26. The method of any one of claims 1 to 25, further comprising ablating afferent nerves in the thoracic dorsal root ganglion.

27. The method of claim 26, wherein ablating afferent nerves in the thoracic dorsal root ganglion supports palliative ventilation therapy.

28. The method of any one of claims 1 to 27, wherein chemical ablation or radiofrequency ablation is administered once in a single dose.

29. The method of any one of claims 1 to 27, wherein chemical ablation or radiofrequency ablation is administered periodically.

30. The method of any one of claims 1 to 29, wherein the subject has pulmonary inflammatory disease.

31. The method of any one of claims 1 to 30, wherein the subject has a neurogenic inflammatory process occurring in a lung.

32. A neurolytic agent or radiofrequency source for use in the method of any one of claims 1 to 31.

33. Use of a neurolytic agent or radiofrequency source for the manufacture of a medicament for use in the method of any one of claims 1 to 31.
